(19) United States
(12) Patent Application Publication
(10) Pub. No.: US 2022/0225913 A1
(43) Pub. Date: Jul. 21, 2022

(54) Blood Draw Assembly and Related Devices and Methods

(71) Applicant: Becton, Dickinson and Company, Franklin, NJ (US)

(72) Inventor: Jithendra Kumar Sathyanarayana Naidu, Singapore (SG)

(21) Appl. No.: 17/575,809

(22) Filed: Jan. 14, 2022

Related U.S. Application Data
(60) Provisional application No. 63/138,169, filed on Jan. 15, 2021.

(51) Int. Cl. A61B 5/15

(52) U.S. Cl. CPC A61B 5/150496; A61B 5/15003; A61B 5/150389; A61B 5/150732

(57) ABSTRACT

A needle adapter may include a body, which may include a distal end and proximal end. The needle adapter may include a needle extending through the body. The needle may include a distal tip and a proximal tip. The distal tip may extend distal to the distal end of the body. The proximal tip may extend proximal to the proximal end of the body. The needle assembly may include a sheath coupled to the body and extending over the proximal tip. The needle assembly may include a hydrophobic vent element surrounding the body. The needle assembly may include a septum coupled to the distal end of the body.

### CROSS-REFERENCE TO RELATED APPLICATION

The present application claims priority to U.S. Provisional Application Ser. No. 63/138,169, entitled “Blood Draw Assembly and Related Devices and Methods”, filed Jan. 15, 2021, the entire disclosure of which is hereby incorporated by reference in its entirety.

### BACKGROUND

A catheter is commonly used to infuse fluids into vasculature of a patient. For example, the catheter may be used for infusing normal saline solution, various medicaments, or total parenteral nutrition.

The catheter may include a peripheral intravenous (“IV”) catheter. In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter system. Once placement of the introducer needle has been confirmed, the clinician may remove the introducer needle, leaving the catheter in place for future fluid infusion.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to a blood draw assembly and related devices and methods. In some embodiments, a blood draw assembly may include a needle adapter, which may include a body. In some embodiments, the body may include a distal end and proximal end. In some embodiments, the needle adapter may include a needle extending through the body. In some embodiments, the needle may include a distal tip and a proximal tip. In some embodiments, the distal tip may extend distal to the distal end of the body. In some embodiments, the proximal tip may extend proximal to the proximal end of the body.

In some embodiments, the needle adapter may include a sheath coupled to the body and extending over the proximal tip. In some embodiments, the needle adapter may include a hydrophobic vent element, which may surround the body. In some embodiments, the needle adapter may include a septum coupled to the distal end of the body.

In some embodiments, the blood draw assembly may include a Y-adapter, which may include a first proximal port, a second proximal port, and a distal port. In some embodiments, the needle adapter may be coupled to the first proximal port. In some embodiments, the septum may be aligned with the second proximal port. In some embodiments, an air vent pathway may extend proximally along an outer edge of the septum, through the hydrophobic vent element, between an outer surface of the body and an inner surface of the first proximal port, and out the first proximal port.

In some embodiments, the needle may include a notch. In some embodiments, the body may include a notch aligned with the notch of the needle. In some embodiments, the hydrophobic vent element may contact an outer edge of the notch of the body. In some embodiments, a blood flow pathway extends through the needle and through the notch in the body to the hydrophobic vent element.

In some embodiments, the body may include a collar disposed between the distal end of the body and the proximal end of the body. In some embodiments, the collar may include threading. In some embodiments, the air vent pathway may extend between an inner surface of the collar and an outer surface of the first proximal port. In some embodiments, the blood draw assembly may include an extension tube integrated with the distal port. In some embodiments, the blood draw assembly may include a catheter adapter and a catheter extending distally from the catheter. In some embodiments, a distal end of the extension tube may be integrated within a catheter adapter. In some embodiments, a proximal end of the extension tube may be integrated within the distal port.

In some embodiments, the hydrophobic vent element may contact a proximal end of the septum and a stepped surface in the body. In some embodiments, the body may include a catch feature configured to pull the septum. In some embodiments, the body may include a collar disposed between the distal end of the body and the proximal end of the body. In some embodiments, an outer diameter of the hydrophobic vent element may be greater than an outer diameter of the septum.

In some embodiments, the blood draw assembly may include a guidewire extending through the needle. In some embodiments, a distal end of the guidewire may include a groove spaced apart from a distal tip of the guidewire. In some embodiments, the guidewire may be configured to rotate.

In some embodiments, the hydrophobic vent element may be disposed within a wall of the body and in fluid communication with the notch. In some embodiments, the septum may be secured to the distal end of the body. In some embodiments, the septum may be secured to the distal end of the body via adhesive. In some embodiments, the septum may be proximal to the second proximal port. In some embodiments, a proximal end of the septum is flush with a proximal end of the first proximal port.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of a prior art blood draw assembly;

FIG. 1B is a cross-sectional view of a portion of the prior art blood draw assembly;

FIG. 2A is an upper perspective view of an example blood draw assembly, illustrating an example needle adapter, according to some embodiments;

FIG. 2B is an upper perspective view of the needle adapter of FIG. 2A, according to some embodiments;

FIG. 2C is an exploded view of the needle adapter of FIG. 2A, according to some embodiments;

FIG. 2D is a cross-sectional view of the needle adapter of FIG. 2A coupled to an example Y-adapter, illustrating an example air vent pathway, according to some embodiments;

FIG. 2E is an enlarged cross-sectional view of a portion of the needle adapter of FIG. 2A, according to some embodiments;

FIG. 2F is a cross-sectional view of the needle adapter of FIG. 2A coupled to the Y-adapter, illustrating an example blood flow pathway, according to some embodiments;

FIG. 2G is an enlarged cross-sectional view of a portion of the needle adapter of FIG. 2A, according to some embodiments;

FIG. 3A is an upper perspective view of the needle adapter of FIG. 2A and an example guidewire, according to some embodiments;

FIG. 3B is an upper perspective view of an example distal end of the guidewire, according to some embodiments;

FIG. 4A is an upper perspective view of the blood draw assembly, illustrating another example needle adapter, according to some embodiments;

FIG. 4B is an upper perspective view of the needle adapter of FIG. 4A coupled to the Y-adapter, according to some embodiments;

FIG. 4C is an upper perspective view of the needle adapter of FIG. 4A, according to some embodiments;

FIG. 4D is an exploded view of the needle adapter of FIG. 2A, according to some embodiments;

FIG. 4E is a cross-sectional view of the needle adapter of FIG. 4A coupled to the Y-adapter, according to some embodiments;

FIG. 4F is an upper perspective view of the needle adapter of FIG. 4A removed from the Y-adapter, according to some embodiments;

FIG. 5A is an upper perspective view of the blood draw assembly, illustrating another example needle adapter, according to some embodiments;

FIG. 5B is an upper perspective view of the needle adapter of FIG. 5A, according to some embodiments;

FIG. 5C is an upper perspective view of the needle adapter of FIG. 5A removed from the blood draw assembly, according to some embodiments;

FIG. 5D is a cross-sectional view of the needle adapter of FIG. 5A removed from an example straight luer adapter, according to some embodiments; and

FIG. 5E is a cross-sectional view of the needle adapter of FIG. 5A coupled to the straight luer adapter, according to some embodiments.

### DESCRIPTION OF EMBODIMENTS

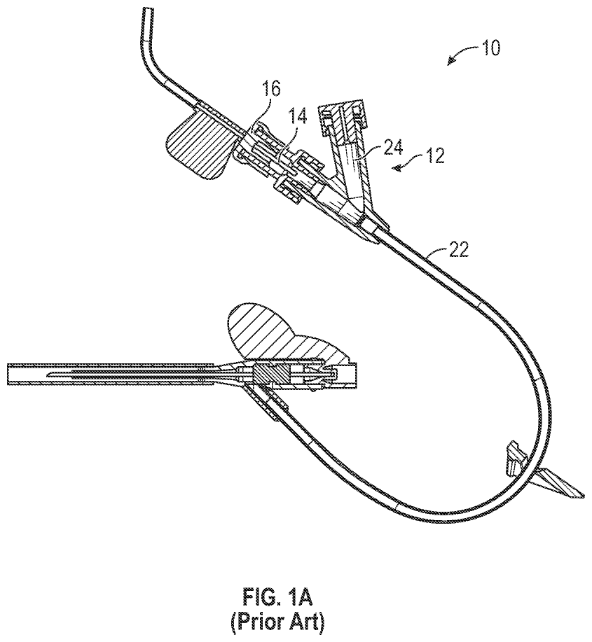

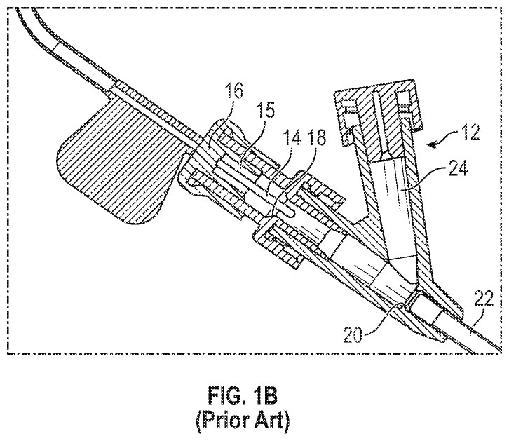

Referring now to FIGS. 1A-1B, a prior art blood draw assembly 10 is illustrated. The prior art blood draw assembly 10 includes a prior art needle adapter 12, which tends to accumulate blood traces after blood draw through the prior art needle adapter 12. The blood traces are difficult to flush and make the prior art needle adapter 12 prone to microbial growth. The blood traces occur in several locations, including in a space 15 between a cannula 14 and a septum 16, at a curved or angled portion 18 of an inner surface of the prior art needle adapter 12, and a stepped portion 20 of the inner surface of the inner surface proximate an extension tube 22. The prior art needle adapter 12 also includes a dead space 24 created due to back pressure, and vein confirmation or blood flashback is often not be clear due to back pressure. Moreover, the prior art needle adapter 12 facilitates mixing of priming fluid and pure blood.

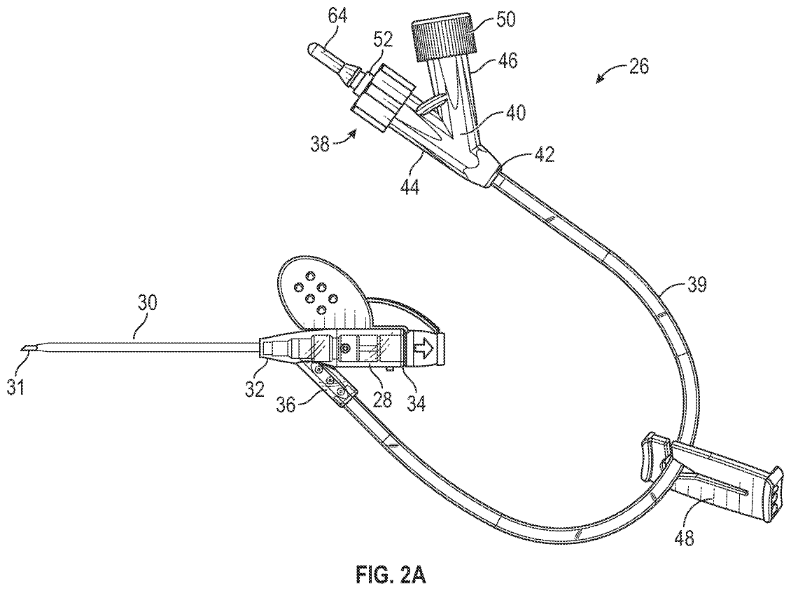

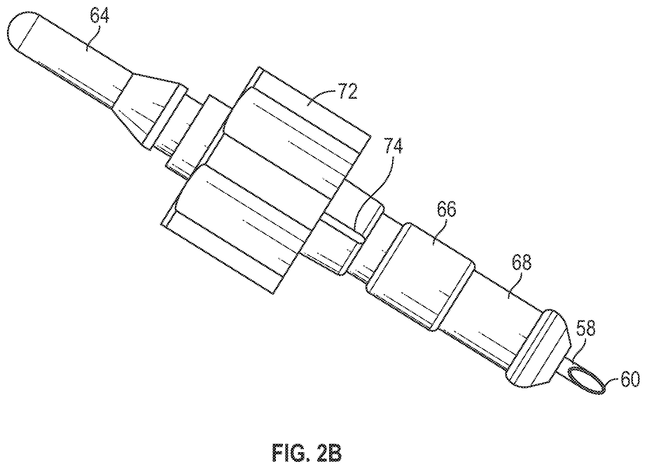

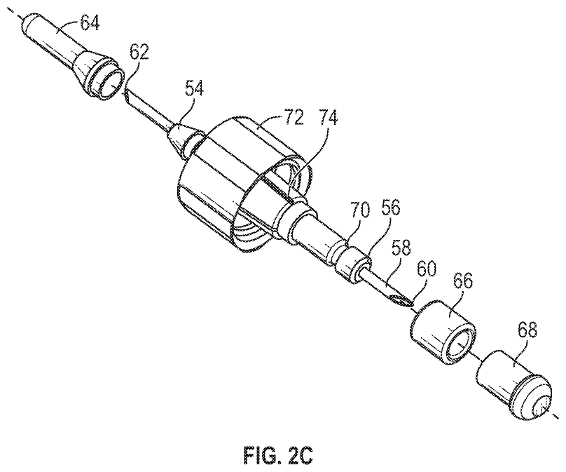

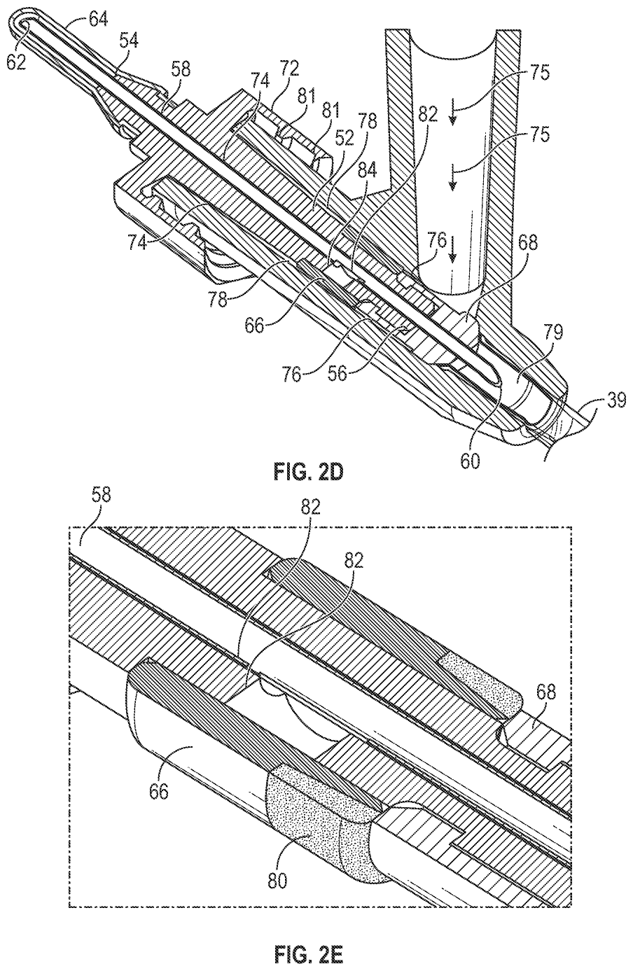

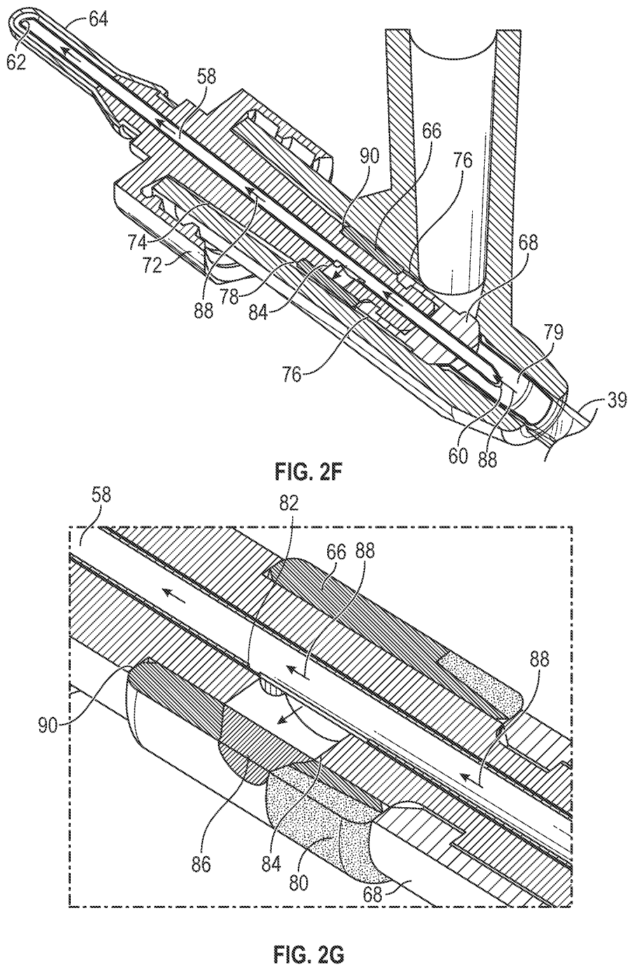

Referring now to FIG. 2A, a blood draw assembly 26 is illustrated, according to some embodiments. In some embodiments, the blood draw assembly 26 may include a catheter assembly, which may include a catheter adapter 28 and a catheter 30 extending distally from the catheter 30. In some embodiments, an introducer needle 31 coupled to a needle hub may extend through the catheter 30 and may be removed after insertion of the catheter 30 into the vasculature and prior to blood draw or collection. In some embodiments, the catheter 30 may include a peripheral intravenous catheter, a central catheter, or a peripherally inserted midline catheter. In some embodiments, the catheter assembly may include or correspond to any suitable catheter assembly, such as, for example, the BD NEXIVA™ Closed IV Catheter system or the BD SAF-T-INTIMA™ Closed IV Catheter System, available from Becton Dickinson & Company of Franklin Lakes, N.J., or another suitable catheter assembly.

In some embodiments, the catheter adapter 28 may include a distal end 32, a proximal end 34, and a lumen extending between the distal end 32 and the proximal end 34. In some embodiments, the catheter adapter 28 may include a side port 36 disposed between the distal end 32 and the proximal end 34.

In some embodiments, the blood draw assembly 26 may include a needle adapter 38, which may be coupled to a Y-adapter 40 of the blood draw assembly 26. In some embodiments, the needle adapter 38 may be pre-attached to the Y-adapter 40. In some embodiments, the Y-adapter 40 may include a distal port 42, a first proximal port 44, and a second proximal port 46. In some embodiments, the Y-adapter may be replaced by an adapter that includes more ports than the distal port 42, the first proximal port 44, the second proximal port 46.

In some embodiments, the blood draw assembly 26 may include an extension tube 39, which may include a distal end integrated with the side port 36 and a proximal end integrated with the distal port 42. In some embodiments, a clamp 48 may be disposed on the extension tube 39 and may move from an unclamped position to a clamped position to prevent blood flow through the extension tube 39. In some embodiments, the needle adapter 38 may be coupled to the first proximal port 44 and a vent cap 50 configured to vent air from the blood draw assembly 26.

Referring now to FIG. 2B-2C, in some embodiments, the needle adapter 38 may include a body 52. In some embodiments, the body 52 may include a distal end 54 and proximal end 56. In some embodiments, the needle adapter 38 may include a needle 58 extending through the body 52. In some embodiments, the needle 58 may include a distal tip 60 and a proximal tip 62. In some embodiments, the distal tip 60 may extend distal to the distal end 52 of the body 54. In some embodiments, the proximal tip 62 may extend proximal to the proximal end 56 of the body 52.

In some embodiments, the needle adapter 38 may include a sheath 64 coupled to the body 52 and extending over the proximal tip 62, which may be sharp. In some embodiments, the sheath 64 may be rubber and/or elastomeric. In some embodiments, in response to coupling a blood collection device to the needle adapter 38, the proximal tip 62 may pierce the sheath 64, the sheath 64 may be compressed towards the body 52, and the needle 58 may be inserted into the blood collection device. In some embodiments, the blood collection device may include a blood collection tube or the BD VACUTAINER® Blood Collection Tube, available from Becton Dickinson & Company of Franklin Lakes, N.J.

In some embodiments, the needle adapter 38 may include a hydrophobic vent element 66. In some embodiments, the hydrophobic vent element 66 may include a ring and may surround the body 52. In some embodiments, the hydrophobic vent element 66 may include a membrane. In some embodiments, the hydrophobic vent element 66 may be configured to pass air. In some embodiments, the hydrophobic vent element 66 may absorb fluid and/or prevent fluid from passing through the hydrophobic vent element 66.

In some embodiments, the needle adapter 38 may include a septum 68, which may be coupled to the distal end 54 of the body 52. In some embodiments, the body 52 may include a catch feature 70 configured to pull the septum 68 in response to removal of the needle adapter 38 from the Y-adapter 40. In some embodiments, the catch feature 70 may include a groove or an annular groove. In some embodiments, the septum 68 may include a protrusion, which may fit inside the catch feature 70. In some embodiments, the distal tip 60 may be disposed distal to the septum 68.

In some embodiments, the body 52 may include a collar 72 disposed between the distal end 54 of the body 52 and the proximal end 56 of the body 52. In some embodiments, an outer surface of the body 52 may include a radial groove 74 to provide an air channel.

Referring now to FIGS. 2D-2E, in some embodiments, the needle adapter 38 may be coupled to the first proximal port 44, as illustrated, for example, in FIG. 2D. In some embodiments, the septum 68 may be aligned with the second proximal port 46. In some embodiments, the distal end of the septum 68 may include a larger diameter than a proximal end of the septum 68. In some embodiments, a distal end of the septum 68 may form a seal with an inner surface of the Y-adapter 40 such that priming fluid 75 flowing proximally through the second proximal port 46 is prevented from flowing distally beyond the septum 68. In some embodiments, a proximal end of the septum 68 may be spaced apart from the inner surface of the Y-adapter 40 to form a clearance 76 through which priming fluid 75 flowing proximally through the second proximal port 46 may flow until it reaches the hydrophobic vent element 66. In some embodiments, the clearance 76 may be annular.

In some embodiments, an air vent pathway may extend proximally along an outer edge of the septum through the clearance 76, proximally through the hydrophobic vent element 66, into a space 78 between an outer surface of the body 52 and an inner surface of the first proximal port 44, through the radial groove 74, and out the first proximal port 44. In some embodiments, the space 78 may be annular. In some embodiments, air may flow through the air vent pathway in response to priming fluid 75 flowing proximally through the second proximal port 46.

In some embodiments, the distal tip 60 of the needle 58 may be disposed within a metal bushing 79 when the needle adapter 38 is coupled to the first proximal port 44. In some embodiments, the septum 68 may provide a seal where the septum 68 contacts the metal bushing 79. In some embodiments, the seal may be annular such that fluid is prevented from moving distal to the distal end of the septum 68.

As illustrated in FIG. 2E, a portion 80 of the hydrophobic vent element 66 may absorb the priming fluid 75 and prevent passage of the priming fluid 75 through the hydrophobic vent element 66. In some embodiments, the portion 80 may be larger or smaller than the portion 80 illustrated in FIG. 2E. In some embodiments, an inner surface of the collar 72 may include threading 81. In some embodiments, the air vent pathway may extend between the inner surface of the collar 72 and an outer surface of the first proximal port 44.

Referring now to FIGS. 2F-2G, in some embodiments, the needle 58 may include a notch 82. In some embodiments, the body 52 may include a notch 84 aligned with the notch 82 of the needle 58. In some embodiments, the hydrophobic vent element 66 may contact an outer edge of the notch 82 of the body 52 and seal the notch 82. In some embodiments, a blood flow pathway extends through the needle 58 and through the notch 82 in the body 52 to the hydrophobic vent element 66. In some embodiments, a portion 86 of the hydrophobic vent element 66 may absorb blood 88. In some embodiments, the portion 86 may be larger or smaller than the portion 86 illustrated in FIG. 2H. In some embodiments, blood 88 may flow proximally through the blood flow pathway in response to insertion of the catheter 30 into vasculature of the patient and/or unclamping of the clamp 48 (see for example, FIG. 2A).

In some embodiments, the hydrophobic vent element 66 may contact the proximal end of the septum 68 and/or a stepped surface 90 in the body 52. In some embodiments, the hydrophobic vent element 66 may thus be secured between the proximal end of the septum 68 and the stepped surface 90, which may be annular. In some embodiments, an outer diameter of the hydrophobic vent element 66 may be greater than an outer diameter of the septum 68 such that the clearance 76 ends. In some embodiments, the hydrophobic vent element 66 may contact the inner surface of the Y-adapter 40.

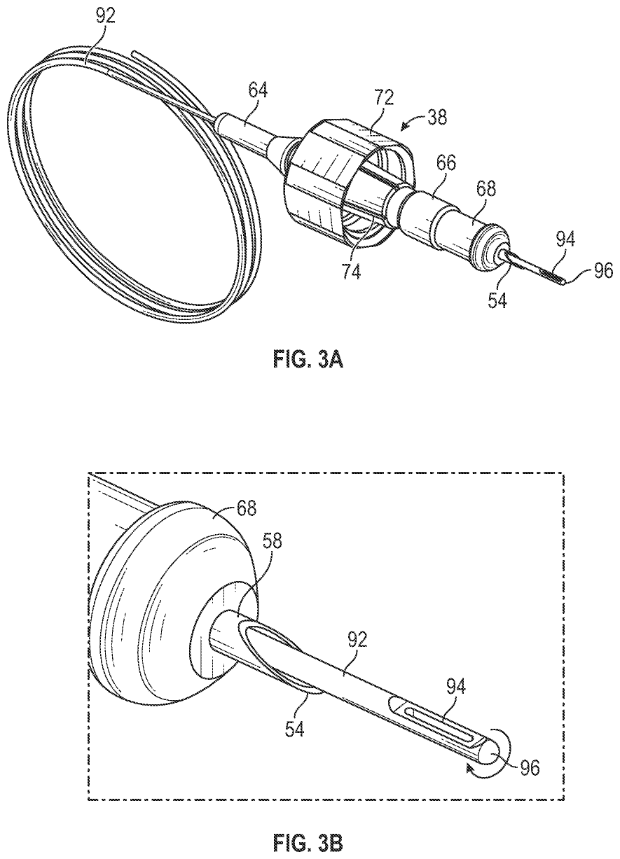

Referring now to FIGS. 3A-3B, in some embodiments, the blood draw assembly 26 may include a guidewire 92, which may extend through the needle 58. In some embodiments, the guidewire 92 may be threaded through the needle 58 and the blood draw assembly 26, which may facilitate blood draw through an occluded catheter. In some embodiments, a distal end of the guidewire 92 may include a groove 94 spaced apart from a distal tip 96 of the guidewire 92. In some embodiments, the guidewire 92 may be configured to rotate, which may facilitate removal of a fibrin sheath inside and/or near a distal end of the catheter 30 (see, for example, FIG. 2A). In some embodiments, the groove 94 may facilitate pulling and/or rotation of the fibrin sheath. In some embodiments, the groove 94 may be disposed within a planar cut at the distal end of the guidewire 92.

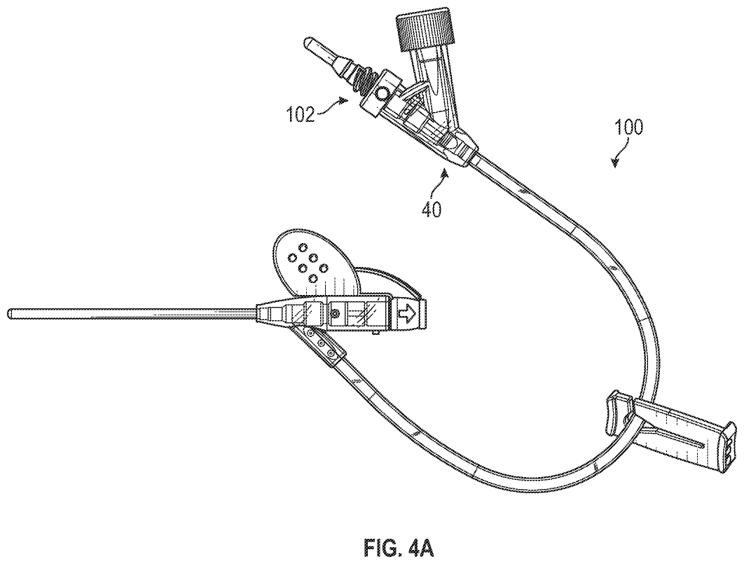

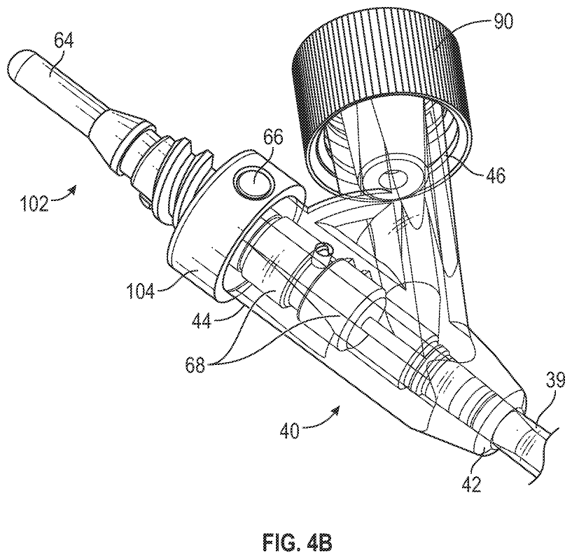

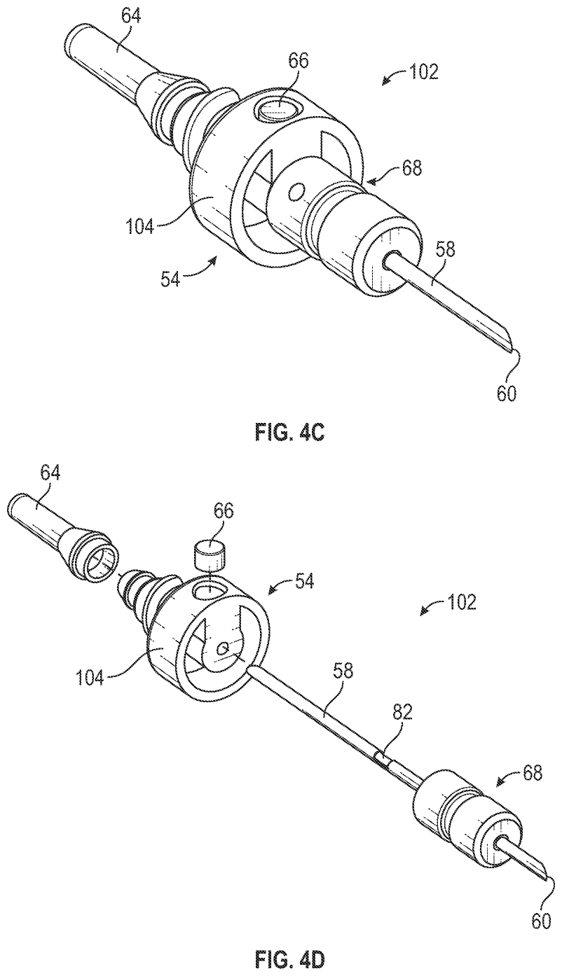

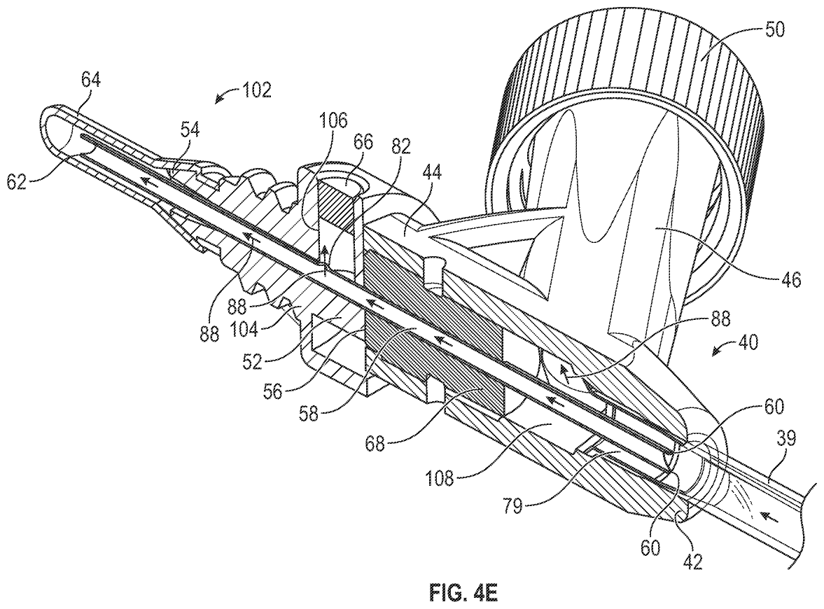

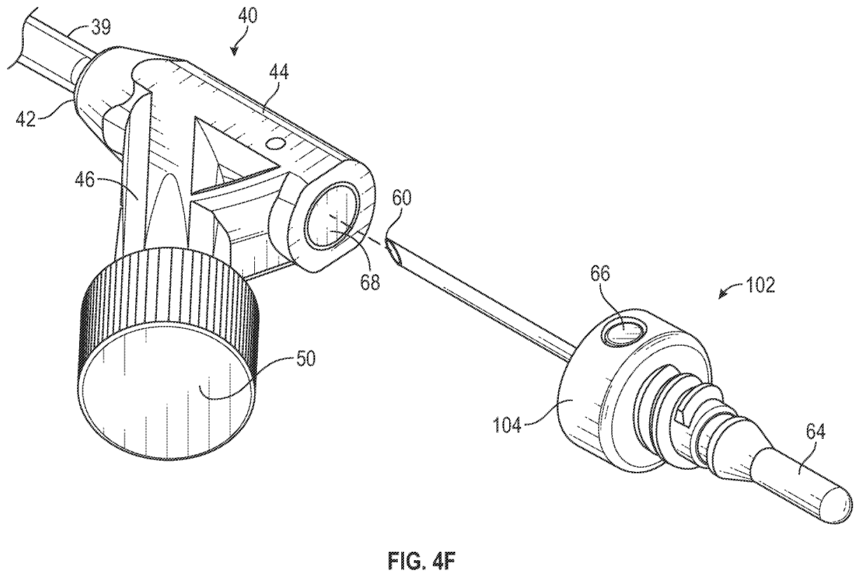

Referring now to FIG. 4A, a blood draw assembly 100 and a needle adapter 102 are illustrated, according to some embodiments. In some embodiments, the blood draw assembly 100 and the needle adapter 102 may be similar or identical to the blood draw assembly 26 and the needle adapter 38, respectively, of FIGS. 2A-3B in terms of one or more components and/or operation. In some embodiments, the needle adapter 102 may be pre-attached to the Y-adapter 40.

Referring now to FIGS. 4B-4E, in some embodiments, the hydrophobic vent element 66 may be disposed within a wall 104 of the body 52 and in fluid communication with the notch 82. In some embodiments, the septum 68 may be secured to the distal end 54 of the body 52. In some embodiments, the septum 68 may be secured to the distal end 54 of the body 52 via adhesive or another suitable attachment means. In some embodiments, the septum 68 may be proximal to the second proximal port 46.

In some embodiments, a blood flow pathway may extend proximally through the needle 58. In some embodiments, the blood flow pathway may also extend through the notch 82 in the needle 58 and into a tunnel 106 in the body 52 to the hydrophobic vent element 66. In some embodiments, the tunnel 106 may extend from the needle 58 through the wall 104 of the body 52. In some embodiments, the tunnel 106 may provide enhanced flashback for vein confirmation.

In some embodiments, in response to blood flowing proximally through the blood flow pathway, air may flow out the hydrophobic vent element 66. In some embodiments, the hydrophobic vent element 66 may be configured to pass air and not liquid, such as blood. In some embodiments, the vent cap 50 may be coupled to the second proximal port 46. In some embodiments, the blood flow pathway may also extend into the second proximal port 46, and air may flow proximally out the vent cap 50. In some embodiments, the blood may flow proximally through the blood flow pathway in response to insertion of the catheter 30 into vasculature of the patient and/or unclamping of the clamp 48 (see for example, FIG. 4A).

In some embodiments, in response to coupling a blood collection device to the needle adapter 102, the proximal tip 62 may pierce the sheath 64, the sheath 64 may be compressed towards the body 52, and the needle 58 may inserted into the blood collection device. In some embodiments, the blood collection device may include a blood collection tube or the BD VACUTAINER® Blood Collection Tube, available from Becton Dickinson & Company of Franklin Lakes, N.J.

In some embodiments, the distal tip 60 may be sharp, which may facilitate insertion of the needle 58 through the septum 68. In some embodiments, the distal tip 60 may be blunt. In some embodiments, the distal tip 60 of the needle 58 may be disposed within the metal bushing 79, which may prevent damage to the inner surface of the Y-adapter. In some embodiments, the Y-adapter may include a low or zero dead space zone 108, which may facilitate flushing and removal of traces of blood. In some embodiments, the low or zero dead space zone 108 may extend between the distal end of the septum 68 and the metal bushing 79.

Referring now to FIG. 4F, in some embodiments, a proximal end of the septum 68 may be flush with a proximal end of the first proximal port 46, which may facilitate cleaning and disinfection of the septum 68. FIG. 4F illustrates removal of the needle adapter 102 from the Y-adapter 40 after completion of blood draw from the patient. In some embodiments, a winged needle set may be inserted through the septum 68 after the needle adapter 102 is removed from the Y-adapter 40. In some embodiments, the vent cap 50 may be removed and replaced with a needle-free luer connector.

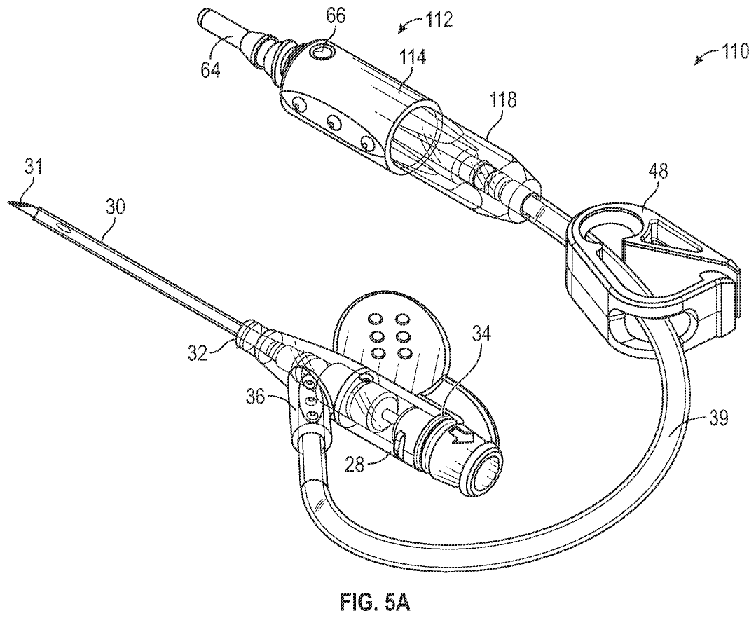

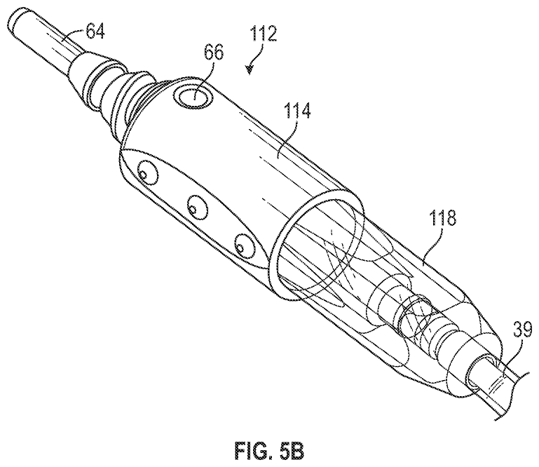

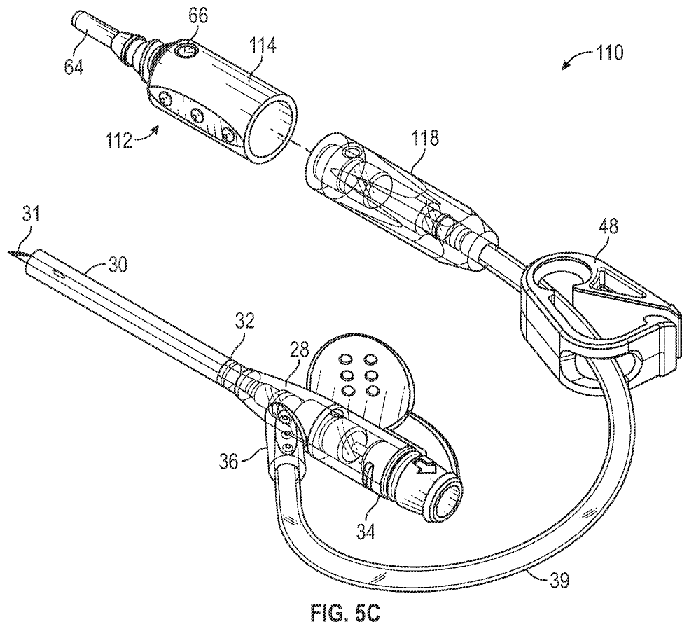

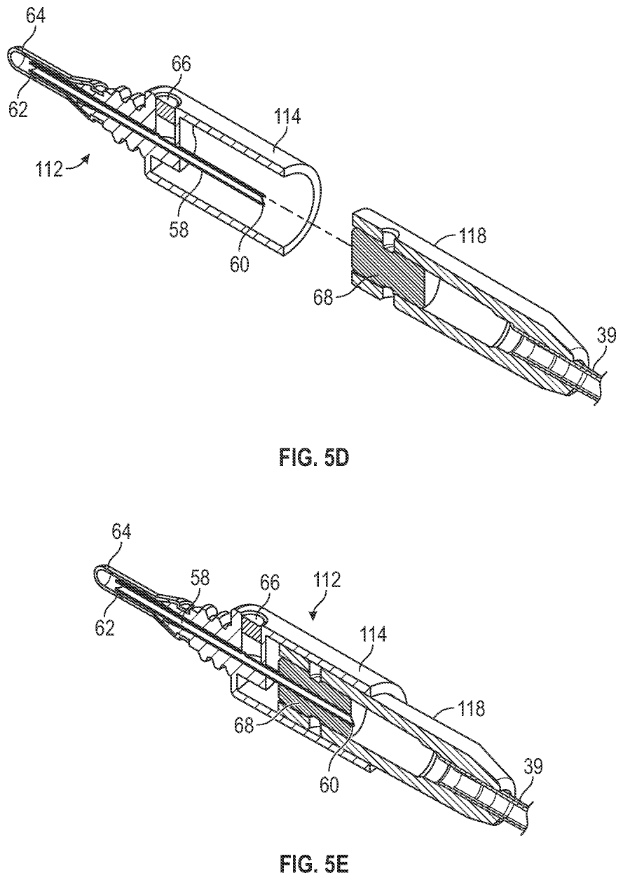

Referring now to FIG. 5A, a blood draw assembly 110 and a needle adapter 112 are illustrated, according to some embodiments. In some embodiments, the blood draw assembly 110 may be similar or identical to the blood draw assembly 26 of FIGS. 2A-3B and/or the blood draw assembly 100 of FIGS. 4A-4F in terms of one or more components and/or operation. In some embodiments, the needle adapter 112 may be similar or identical to the needle adapter 38 of FIGS. 2A-3B and/or the needle adapter 102 of FIGS. 4A-4F in terms of one or more components and/or operation.

In some embodiments, the needle adapter 112 may be pre-attached to the Y-adapter 40. In some embodiments, the body 52 may include a cylindrical portion 114, which may extend distal to the distal tip 60. In some embodiments, the cylindrical portion 114 may act as a needle guard to prevent an accidental stick with the distal tip 60.

In some embodiments, the body 52 may be coupled to a straight luer adapter 118, which may include the septum 68. In some embodiments, extension tube 39 may be integrated within the straight luer adapter 118. In some embodiments, the straight luer adapter 118 may be configured to insert into the cylindrical portion 114.

In some embodiments, the catheter assembly, which may include the catheter 30 and the catheter adapter 28, may be closed and no priming may be necessary. In some embodiments, the extension tube 39 may be shorter, such as, for example, between 1 and 4 inches.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A needle adapter, comprising:
a body comprising a distal end and proximal end;
a needle extending through the body and comprising a distal tip and a proximal tip, wherein the distal tip extends distal to the distal end of the body, wherein the proximal tip extends proximal to the proximal end of the body;
a sheath coupled to the body and extending over the proximal tip;
a hydrophobic vent element surrounding the body; and
a septum coupled to the distal end of the body.

2. The needle adapter of claim 1, wherein the needle comprises a notch, wherein the body comprises a notch aligned with the notch of the needle, wherein the hydrophobic vent element contacts an outer edge of the notch of the body.

3. The needle adapter of claim 1, wherein the hydrophobic vent element contacts a proximal end of the septum and a stepped surface in the body.

4. The adapter of claim 1, wherein the body comprises a catch feature configured to pull the septum.

5. The needle adapter of claim 1, wherein the body further comprises a collar disposed between the distal end of the body and the proximal end of the body.

6. The needle adapter of claim 5, wherein the collar comprises threading.

7. The needle adapter of claim 1, wherein an outer diameter of the hydrophobic vent element is greater than an outer diameter of the septum.

8. The needle adapter of claim 1, further comprising a guidewire extending through the needle.

9. The needle adapter of claim 8, wherein a distal end of the guidewire comprises a groove spaced apart from a distal tip of the guidewire, wherein the guidewire is configured to rotate.

10. A blood draw assembly, comprising:
a needle adapter, comprising:
a body comprising a distal end and proximal end;
a needle extending through the body and comprising a distal tip and a proximal tip, wherein the distal tip extends distal to the distal end of the body, wherein the proximal tip extends proximal to the proximal end of the body;
a sheath coupled to the body and extending over the proximal tip;
a hydrophobic vent element surrounding the body; and
a septum coupled to the distal end of the body;
a Y-adapter comprising a first proximal port, a second proximal port, and a distal port, wherein the needle adapter is coupled to the first proximal port, wherein the septum is aligned with the second proximal port, wherein an air vent pathway extends proximally along an outer edge of the septum, through the hydrophobic vent element, between an outer surface of the body and an inner surface of the first proximal port, and out the first proximal port.

11. The blood draw assembly of claim 10, wherein the needle comprises a notch, wherein the body comprises a notch aligned with the notch of the needle, wherein the hydrophobic vent element contacts an outer edge of the notch of the body.

12. The blood draw assembly of claim 10, wherein a blood flow pathway extends through the needle and through the notch in the body to the hydrophobic vent element.

13. The blood draw assembly of claim 10, wherein the body further comprises a collar disposed between the distal end of the body and the proximal end of the body, wherein the air vent pathway extends between an inner surface of the collar and an outer surface of the first proximal port.

14. The blood draw assembly of claim 10, further comprising an extension tube integrated with the distal port.

15. The blood draw assembly of claim 10, wherein the blood draw assembly further comprises a catheter adapter and a catheter extending distally from the catheter, wherein a distal end of the extension tube is integrated within a catheter adapter, wherein a proximal end of the extension tube is integrated within the distal port.

16. A blood draw assembly, comprising:
a needle adapter, comprising:
a body comprising a distal end and proximal end;
a needle extending through the body and comprising a distal tip, a proximal tip, and a notch disposed between the distal tip and the proximal tip, wherein the distal tip extends distal to the distal end of the body, wherein the proximal tip extends proximal to the proximal end of the body;
a sheath coupled to the body and extending over the proximal tip; and
a hydrophobic vent element disposed within a wall of the body and in fluid communication with the notch.

17. The blood draw assembly of claim 16, further comprising a septum secured to the distal end of the body.

18. The blood draw assembly of claim 17, wherein the septum is secured to the distal end of the body via adhesive.

19. The blood draw assembly of claim 16, further comprising a Y-adapter comprising a first proximal port, a second proximal port, and a distal port, wherein the needle adapter is coupled to the first proximal port, wherein the septum is proximal to the second proximal port.

20. The blood draw assembly of claim 16, wherein a proximal end of the septum is flush with a proximal end of the first proximal port.
